(19) United States
(12) Patent Application Publication
(10) Pub. No.: US 2007/0112277 A1
(43) Pub. Date: May 17, 2007

(54) APPARATUS AND METHOD FOR THE MEASUREMENT AND MONITORING OF BIOELECTRIC SIGNAL PATTERNS

(76) Inventors: Russell J. Fischer, Bernardsville, NJ (US); Joseph Ferraro, Robbinsville, NJ (US); Prince Lal, Stamford, CT (US); Hugh Lusted, Brownsville, CA (US)

Correspondence Address: PATENT DOCKET ADMINISTRATOR;LOWENSTEIN SANDLER P.C., 65 LIVINGSTON AVENUE, ROSELAND, NJ, 07068, US

(21) Appl. No.: 11/549,862

(22) Filed: Oct. 16, 2006

Related U.S. Application Data
(60) Provisional application No. 60/726,910, filed on Oct. 14, 2005.
(60) Provisional application No. 60/726,895, filed on Oct. 14, 2005.
(60) Provisional application No. 60/727,154, filed on Oct. 14, 2005.

(51) Int. Cl. A61B 5/04; A61B 5/00

(52) U.S. Cl. 600/544; 128/903; 600/559; 600/546

(57) ABSTRACT

A wireless apparatus and method for the measurement and monitoring of bioelectric signal patterns associated with EEG, EOG and EMG readings is provided. The apparatus is comprised of at least one measurement device employing the use of three bioelectric sensing electrodes, wherein at least one of the electrodes is configured for secure placement within the ear canal of an individual under medical surveillance. Acoustic stimulation may be provided directly into the ear canal of the individual via an auditory stimulus emitted from the measurement device for evoking brain activity and the subsequent measurement of bioelectric signal patterns associated with the evoked activity.

### CROSS-REFERENCE TO RELATED APPLICATIONS

This application claims the benefit of U.S. Provisional Patent Application No. 60/726,910, filed Oct. 14, 2005, U.S. Provisional Patent Application No. 60/726,895, filed Oct. 14, 2005, and U.S. Provisional Patent Application No. 60/727,154, filed Oct. 14, 2005, which are all hereby incorporated by reference.

### BACKGROUND OF THE INVENTION

1. Field of the Invention

The present invention relates to the field of medical monitoring. More particularly, the present invention is directed to an apparatus and method for continuous medical monitoring of bioelectric signal patterns employing at least one wireless measurement device having at least one electrode adaptable for insertion within the ear canal.

2. Description of the Related Art

Electroencephalography (EEG), electrooculography (EOG) and electromyography (EMG) are techniques used for measuring, respectively, the electrical patterns corresponding to brain activity, eye movement associated with the resting potential of the retina and muscle contraction. The practice of assessing bioelectric signal patterns associated with the aforementioned techniques are recognized for their roles in a plurality of therapeutic and diagnostic applications.

Assessing bioelectric signal patterns associated with an EEG, EOG and EMG can be very useful in identifying abnormalities and particular areas of impairment related, respectively, to brain function, eye movement and muscle response. For example, a disturbance or known variation in the bioelectric signature of a normal EEG may generally be used to determine the existence of a neurological impairment. Typically, bioelectric signal patterns associated with an EEG reading is used to detect occurrences of seizures, evaluate the extent of trauma and injuries to the brain, diagnose the effects of tumors in particular locations of the brain, identify various infections and degenerative diseases, and evaluate sleeping disorders. The bioelectric signal pattern of an EEG is also frequently used to confirm brain death in a comatose patient. Similarly, bioelectric signal patterns associated with an EMG reading may be useful for assessing a variety of sleeping disorders. For example, the electrical activity related to muscle contractions associated with nighttime bruxism (i.e., dysfunctional clenching and grinding of the teeth) can be diagnosed with proper placement of electrodes.

Bioelectric signal patterns associated with an EOG reading are typically useful for studying and analyzing movements associated with the eye. The eye is the source of a steady electric potential field that can be described as a fixed dipole with positive potential at the cornea and negative potential at the retina. The magnitude of this potential is in the range of 0.4-1.0 mV. The potential is not related to light stimulation and is not generated by excitable tissue, but rather it is attributed to the higher metabolic rate of the retina. This potential difference and the rotation of the eye are the basis for the bioelectric signal pattern associated with an EOG reading. The bioelectric signal pattern associated with an EOG reading is very useful for determining the onset of REM sleep, which is sleep associated with a relatively high degree of eye motion.

Providing a means for assessing bioelectric signal patterns associated with EEG, EOG and EMG readings are not only useful for identifying impairments or abnormalities, but are also tremendously useful for monitoring the effectiveness of rehabilitative measures and progress of recovery. Therefore, when properly assessed, bioelectric signal patterns can be significantly useful for assisting healthcare professionals in determining the most effective treatments and appropriate preventative measures to be implemented.

Modern devices for measuring and monitoring bioelectric signal patterns associated with EEG, EOG and EMG are typically only available in a laboratory setting and operated by trained technicians. However, there is obvious value in being able to provide a means for measuring these bioelectric signal patterns in a home setting for monitoring sleeping disorders, normal daily activities (e.g., work, recreation or operation of a vehicle) or perhaps for determining level of consciousness and cognitive performance of military personnel in the field. However, these particular monitoring applications are impeded by the need to place skin-contact electrodes at various prescribed locations on the body. Correct placement of skin-contact electrodes typically requires some level of training, not only to find the proper locations for electrode application, but also to observe the received signal in order to assure the signal quality associated with placement of the skin-contact electrodes are acceptable. In addition, modern skin-surface electrodes used in the measurement of bioelectric signal patterns are prone to disruption resulting from normal motions of the body. In a sleep study, for example, involuntary motions can disturb skin-surface electrodes typically affixed to the head or neck. Consequently, the resulting bioelectric signal pattern recordings contain artifacts, which thereby complicate analysis.

Moreover, these aforementioned assessment devices and techniques, although non-invasive, require the use of expensive and intricate equipment set-ups. Due to the sophistication and intricacies of these aforementioned assessment techniques, a lab or clinical type setting is typically required and, therefore, there exist obvious limitations on the scope for which these techniques can be used. For example, it is extremely difficult and costly to provide the aforementioned assessment techniques as a means for allowing continuous monitoring of individuals undergoing recovery due to the lack of mobility inherent with such lab-type equipment.

Accordingly, it is desirable to provide an improved apparatus and method for the measurement and monitoring of bioelectric signal patterns associated with EEG, EOG and EMG readings.

### SUMMARY OF THE INVENTION

It is an object of the present invention to provide a minimally invasive bioelectric measurement device employing a lightweight and cost effective design, thereby further providing a less cumbersome and highly mobile means for monitoring bioelectric signal patterns.

It is another object of the present invention to provide a minimally invasive and mobile measurement device configured to provide a means for continuous monitoring of bioelectric signal patterns to evaluate the effectiveness of rehabilitative measures, drug efficacy and the progression of recovery or mental and physical deterioration.

It is another object of the present invention to provide a minimally invasive and mobile measurement device employing electrode sites that elicit the requisite bioelectric signals, have enhanced immunity to motion artifacts, are simple to apply and are comfortable for the wearer.

In light of the foregoing, these and other objects are accomplished in accordance with the principles of the present invention, wherein the novelty of the present invention will become apparent from the following detailed description and appended claims, and wherein a wireless apparatus employing a bioelectric measuring device having at least one electrode positioned within the ear canal and a remote monitoring device for analyzing measured bioelectric related data is provided.

The measurement device employs the use of three electrodes, wherein at least one of these electrodes is configured for positioning within the car canal of an individual under medical surveillance. Electrodes positioned within the ear canal provide exceptional contact, as well as robust measurement of bioelectric signal patterns associated with EEG, EOG and EMG readings. A plurality of alternative configurations are presented for the positioning of electrodes in close proximity to the ear canal, thereby providing optimal bioelectric measurement points for various medical applications. The bioelectric measurement device of the present invention is also configured to present acoustic stimulation directly into the ear canal for evoking brain activity and the subsequent measurement of bioelectric signal patterns associated with the evoked activity.

At least one remote monitoring device is configured to wirelessly receive and analyze bioelectrical signal patterns measured by the bioelectric measuring device. The remote monitoring device is further configured to detect abnormalities and execute predefined notification procedures in response to the detections, wherein the notification procedures may include transmission of bioelectric related data to a healthcare professional equipped with a medically enabled mobile device.

### BRIEF DESCRIPTION OF THE DRAWINGS

The above and other objects and advantages of the present invention will be apparent upon consideration of the following detailed description, taken in conjunction with the accompanying drawings, in which like reference characters refer to like parts throughout, and in which:

FIG. 1 is an exemplary block diagram of a wireless apparatus employing a bioelectric measurement device and remote monitoring device in accordance with an embodiment of the present invention.

FIGS. 2A and 2B illustrate opposing side views of an exemplary bioelectric measurement device housing structure in accordance with an embodiment of the present invention.

FIGS. 3A and 3B illustrate an exemplary bioelectric measurement device configured for placement between an auricle of the ear and an adjacent side of a head of an individual in accordance with an embodiment of the present invention.

FIGS. 4A and 4B illustrate exploded views of an exemplary electrode equipped ear canal insert coupled to the housing structure of the bioelectric measurement device illustrated in FIG. 1 and configured for insertion within an ear canal of an individual in accordance with an embodiment of the present invention.

FIGS. 5A and 5B illustrate exploded views of an exemplary electrode equipped ear canal insert containing bioelectric measurement components therein and configured for insertion entirely within an ear canal of an individual in accordance with an embodiment of the present invention.

FIGS. 6A and 6B illustrate an exemplary montage of electrode placement in accordance with an embodiment of the present invention.

FIGS. 7A and 7B illustrate another exemplary montage of electrode placement in accordance with an embodiment of the present invention.

FIGS. 8A and 8B illustrate another exemplary montage of electrode placement in accordance with an embodiment of the present invention.

FIGS. 9A and 9B illustrate another exemplary montage of electrode placement in accordance with an embodiment of the present invention.

FIGS. 10A and 10B illustrate another exemplary montage of electrode placement in accordance with an embodiment of the present invention.

FIG. 11 is a flowchart illustrating the steps employed in monitoring and analyzing bioelectric signal patterns associated with EEG, EOG and EMG readings of an individual in accordance with an embodiment of the present invention.

FIG. 12 is a flowchart illustrating the steps employed in evoking brain activity and monitoring the corresponding bioelectric signal pattern associated with an EEG reading of an individual in accordance with an embodiment of the present invention.

It is to be understood that the above-identified drawing figures are for purposes of illustrating the concepts of the present invention and may not be to scale, and are not intended to be limiting in terms of the range of possible shapes and proportions of the present invention.

### DETAILED DESCRIPTION OF THE PREFERRED EMBODIMENTS

The present invention is directed towards an apparatus and method for the wireless measurement of bioelectric signal patterns employing at least one bioelectric measurement device having at least one electrode adaptable for insertion into the ear canal. For purposes of clarity, and not by way of limitation, illustrative views of the present invention are described with references made to the above-identified drawing figures. Various modifications obvious to one skilled in the art are deemed to be within the spirit and scope of the present invention.

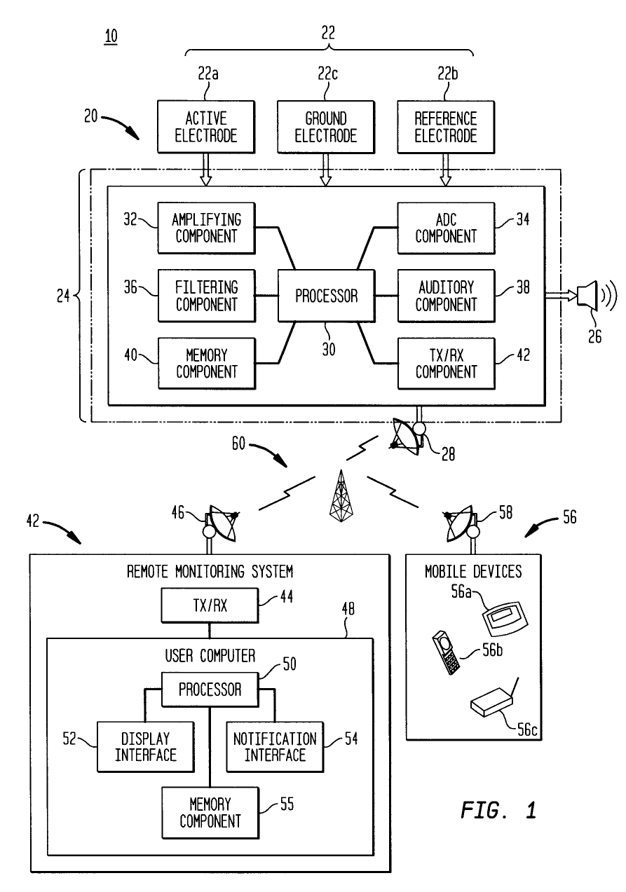

An exemplary wireless apparatus 10 is illustrated in FIG. 1. In accordance with a preferred embodiment of the present invention, apparatus 10 is comprised of a bioelectric measurement system 20, a remote monitoring system 42 and mobile devices 56. Bioelectric measurement system 20 is utilized in connection with a patient undergoing medical surveillance to measure bioelectric signal patterns associated with EEG, EOG and EMG readings. Remote monitoring system 42 and mobile devices 56 are configured to receive transmissions 60 of bioelectric related data from bioelectric measurement system 20. Bioelectric related data may be transmitted via antenna 28 of bioelectric measurement system 20 and received via antennas 46 and 58 of remote monitoring system 42 and mobile devices 56, respectively. Alternatively, bioelectric related data may be transmitted by remote monitoring system 42 and mobile devices 56 to bioelectric measurement system 20. For example, instructions may be provided to system 20 from a healthcare professional via mobile device 56 to induce an acoustic stimulation for purposes of monitoring the resulting brain activity of a patient. It should be noted that bioelectric measurement system 20, remote monitoring system 42 and mobile devices 56 are not limited to use of antennas 28, 46 and 58, but rather are provided as exemplary means for transmitting and receiving transmissions 60 in accordance with the present invention described herein. Alternative transmitting and receiving means may be employed in, and are well within the scope of, the present invention.

Bioelectric measurement system 20 is comprised of electrodes 22, an electronics unit 24, a speaker 26 and an antenna 28. Electronics unit 24 may include processing and wireless transmission components such as a processor 30, an amplifying component 32, an analog-to-digital converter (ADC) component 34, a filtering component 36, an auditory component 38, a memory component 40 and a transceiver component 42. Electrodes 22 and speaker 26 are coupled to signal processing unit 24 of bioelectric measurement system 20.

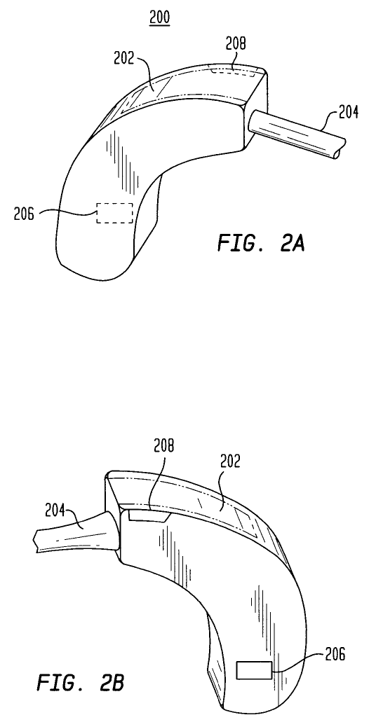

Electrodes 22 includes an active electrode 22a, a reference electrode 22b and a ground electrode 22c, which may be positioned at optimal EEG, EMG and EOG measurement points in the ear canal and at external points in close proximity to the ear (detailed description of various electrode arrangements and placement montages are provided in conjunction with FIGS. 2-10). Speaker 26 may be an electronic device used to transform varying electric current into audible sound, a computer peripheral that reproduces speech and/or music, or any other suitable electro-acoustic transducer that converts electrical signals into sounds for presentation into the auditory canal of an individual undergoing medical surveillance via bioelectric measuring system 20.

Remote monitoring system 42 may be located at various locations suitable for monitoring the bioelectric signal patterns transmitted by bioelectric measuring system 20. In an alternative embodiment, there may be more than one remote monitoring system 42 for monitoring received bioelectric signal patterns. Remote monitoring system 42 is comprised of a transceiver 44 coupled to a user computer 48. User computer 48 is comprised of a processor 50, a display interface 52, a notification interface 54 and a memory component 55. Transceiver 44 is configured to transmit and receive data transmissions 60 via antenna 46 to and from transceiver 42 via antenna 28 of bioelectric measurement system 20.

Mobile devices 56 may include a pager 56a, a cellular or mobile telephone 56b, a personal digital assistant (PDA) 56c or any other suitable mobile device enabled for secure and robust wireless connectivity. Bioelectric related data transmissions 60 may be received at or transmitted from mobile devices 56 via antenna 58. Mobile devices 56 are preferably of the type that are medically enabled. For example, mobile devices 56 may be integrated with a wireless application protocol (WAP) in order to provide secure access to a hospital's computer network via a designated medical web site. Mobile devices 56 may also be equipped to run medically related applications or software. Such medically enabled devices provide healthcare personnel with an ability to work seamlessly regardless of their disparate locations. In addition, mobile devices 56 may provide e-mail and instant messaging services. For example, a doctor equipped with a medically enabled mobile device may receive captured images of an abnormal EEG pattern related to a patient from remote monitoring site 42 and prescribe instructions via the medically enabled mobile device back to remote monitoring site 42 or another medical staff member also equipped with a medically enabled mobile device.

Transceiver 42 of bioelectric measurement system 20, transceiver of remote monitoring system 42 and mobile devices 56 may be transceivers that are compatible with Wi-Fi standard IEEE 802.11, BLUETOOTH™ enabled, a combination of local area network (LAN), wide area network (WAN), wireless area network (WLAN), personal area network (PAN) standards or any other suitable means to permit robust wireless transmission of bioelectric related data. For example, transceiver 42 of bioelectric measurement system 20, transceiver of remote monitoring system 42 and mobile devices 56 may be BLUETOOTH™ enabled, thereby providing a means for connecting and exchanging bioelectric related information between devices such as a personal digital assistants (PDAs), cellular phones, notebook and desktop computers, printers, digital cameras or any other suitable electronic device via a secured short-range radio frequency. It should be noted that the aforementioned are provided merely as exemplary means for wireless transmission of bioelectric related data. Other suitable wireless transmission and receiving means may be employed in the present invention.

Electrical activity associated with EEG, EOG and EMG readings are typically measured using, respectively, skin-surface electrodes on the scalp, skin-surface electrodes at locations on the skin that are vertical or horizontal in position to the eyes and skin-surface electrodes or needles in muscular contraction areas of interest. However, the present invention employs a means for measuring bioelectrical signal patterns associated with EEG, EOG and EMG readings by utilizing electrodes positioned within and in close proximity to an individual's ear canal. Bioelectrical measurements taken via electrodes positioned primarily within the ear canal are advantageous in that the ear canal is in close proximity to the cerebral cortex and the eyes, yet not on the exterior surface of the skin. This particular positioning of electrodes provides for relatively strong bioelectric signal patterns to be recorded, while also providing reduced exposure to disruption of measurements due to motion.

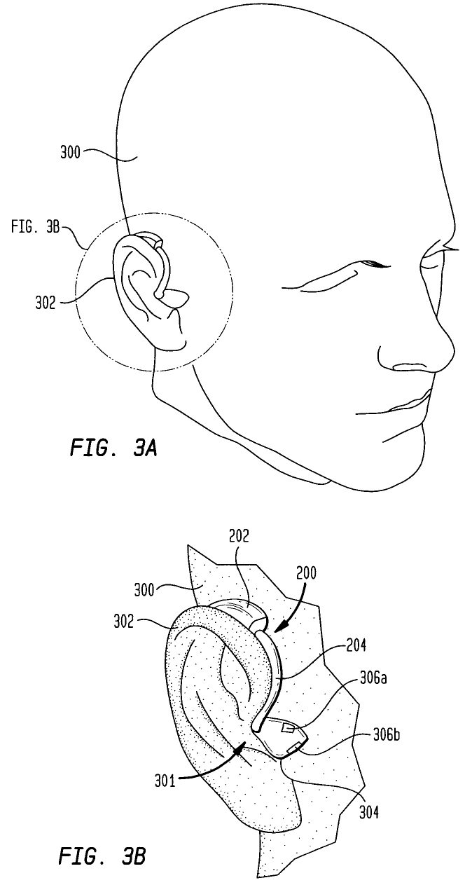

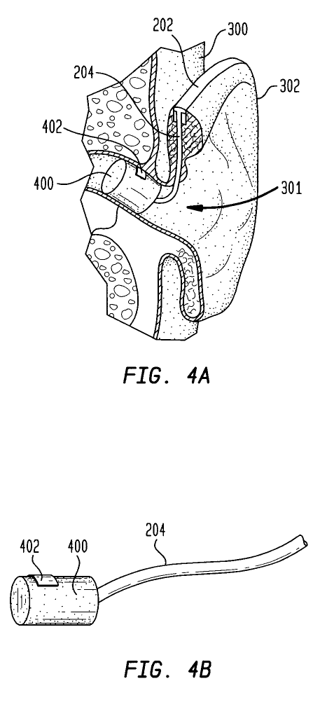

In providing a means for measuring bioelectrical signal patterns associated with EEG, EOG and EMG readings via the ear canal, a hearing aid type device may be employed to house the electronic components necessary to process bioelectric readings received by electrodes 22 and transmit the processed bioelectric readings to remote monitoring system 42 of FIG. 1. Such an exemplary hearing aid type device is described with reference to FIGS. 2A and 2B. A bioelectric measurement device 200 of FIG. 2A may be utilized for measuring bioelectric signal patterns and is comprised of an external housing 202, a flexible processing extension 204 and a moldable ear canal insert 304 or 400, respectively, having electrodes 306a and 306b or electrode 402 affixed thereon (illustrated in corresponding FIGS. 3B-4B). Bioelectric measurement device 200 may also include external electrodes 206 and 208 on the inside surface of housing 202, as depicted in FIG. 2B (for particular applications to be described).

Exterior housing 202 of measurement device 200 is constructed to house electronics unit 24 of FIG. 1, thereby providing a wireless capable processing means for continuous medical surveillance of an ambulatory individual. Flexible processing extension 204 is coupled to a soft ear canal insert having at least one electrode disposed thereon, thereby providing an electrical connection between disposed electrodes and processing unit 24. The body of flexible processing extension 204 is preferably made of a pliable material in order to easily conform and conceal flexible processing extension 204 behind the curvature of the ear.

Affixing electrodes on a soft ear canal insert, as illustrated in FIGS. 3-5, has many advantages over modern bioelectric measurement devices in that the electrodes' locations and spacing in the present invention are predetermined by their affixed positions on the ear canal insert, rather than requiring a healthcare professional to be trained in the proper positioning of electrodes on the external surface of the skin. Therefore, proper electrode locations may be achieved automatically when the electrode outfitted ear canal insert is positioned within the ear canal, thereby significantly simplifying the means for measuring bioelectric signal patterns. Exemplary electrode outfitted ear canal inserts are illustrated in FIGS. 3B-5B and described in the following corresponding detailed description.

A preferred attachment of bioelectric measurement device 200 to an individual under medical surveillance is illustrated in FIGS. 3A-3B. In FIGS. 3A-3B, measurement device 200 is constructed so as to be situated only partially within the ear canal of an individual. For example, as illustrated in FIGS. 3A and 3B, exemplary measurement device 200 is comprised of housing 202, flexible processing extension 204 and electrode outfitted ear canal insert 304. Measurement device 200 is configured for suitable placement between an auricle 302 of the ear and a side of the head 300 of an individual. As illustrated in the enlarged view of FIG. 3B, housing 202 is shaped to the curved contour of the ear of an individual. Flexible processing extension 204 extends from an end of housing 202 to ear canal insert 304, which is inserted within ear canal 301 of the individual.

Ear canal insert 304 is outfitted with at least one electrode. However, for purposes of illustration, and not by way of limitation, ear canal insert 304 of FIG. 3B is shown with two bioelectric sensing electrodes 306a and 306b, spaced about 180 degrees apart on the surface of ear canal insert 304. It should be understood that the number of electrodes provided on ear canal insert 304 may vary depending on the optimal measurement points determined for a particular medical application. For example, in FIGS. 4A-4B, ear canal insert 400 provided within ear canal 301 is outfitted with only one electrode 402.

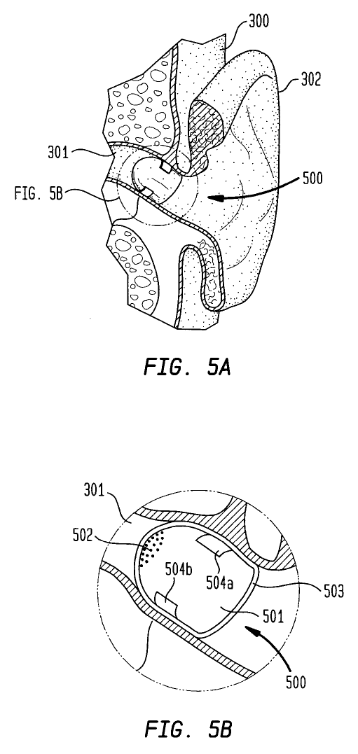

In an alternative embodiment, an exemplary bioelectric measurement device 500, as illustrated in FIGS. 5A-5B, may be provided. Similar to the electrode equipped ear canal inserts of FIGS. 3B-4B, measurement device 500 is constructed and configured to provide a means for inserting a bioelectric sensing electrode within ear canal 301 of an individual. Measurement device 500 is comprised of a housing 501, perforated section 502, moldable exterior shell 503 and bioelectric sensing electrodes 504a and 504b. Rather than have electronics unit 24 provided in a housing structure residing outside the ear canal (as shown in measurement device 200 illustrated in FIGS. 3B-4B), the signal processing and wireless transmission components of electronics unit 24 may all be incorporated entirely within housing 501 of the ear canal shaped insert of measurement device 500.

Perforated section 502 allows for audible sounds to be communicated from measurement device 500 into ear canal 301 of an individual. For example, speaker 16 of FIG. 1 may be positioned adjacent to perforated section 502 of housing 501 in order to provide a means for transmitting audible tones and messages through perforated section 502 and into ear canal 301 for purposes of, for example, evoking brain activity in response to acoustical stimuli.

A moldable exterior shell 503 may be provided circumferentially about the exterior surface of housing 501 of measurement device 500. Moldable exterior shell 503 is preferably constructed of a soft, yet durable, material capable of conforming to the interior walls of an individual's auditory canal in order to provide a comfortable and secure fitting of measurement device 500 within ear canal 301. For example, moldable exterior shell 503 may be constructed of a memory foam that can be compressed and inserted into the auditory canal. When the memory foam is released it expands and provides a secure custom fitting within the individual's auditory canal. It will be understood that the use of a memory foam is only one of many suitable materials that may be used to construct a moldable exterior shell 503 and is merely provided as an example for purposes of illustrating the present invention. In addition, ear canal inserts 304 and 400 illustrated in FIGS. 3B-4B may similarly be constructed with a moldable exterior shell 503 (not shown) to provide a secure custom fitting within the auditory canal of the individual. A soft compressible insert provides the advantage of securely holding electrodes installed on a soft ear canal insert in contact with the surface via the restoring force of the compressible insert.

Electrodes 306a and 306b illustrated in FIG. 3B, electrode 402 illustrated in FIG. 4 and electrodes 504a and 504b illustrated in FIG. 5B may be fabricated from typical materials suitable for bioelectrical measurement. For example, the electrodes may be fabricated from various conductive polymers and metal films. Since the electrodes are, in most circumstances, located within ear canal 301 of an individual via a compressible insert, the need for adhesives and gels, typically used with skin-contact electrodes, are not required.

Although bioelectric signal patterns associated with EEG, EOG and EMG readings can feasibly be measured with the use of only two electrodes, typically referred to as an active electrode and a reference electrode, three electrodes are commonly used. The use of three electrodes allows an individual under medical surveillance to be isolated from ground so that contact with an electric source would not result in the individual creating a path to ground. In addition, three electrodes are preferably used to eliminate electrical noise sources common to both the active and reference electrodes during measurement.

In EEG electrode arrangements utilizing the ear canal, it is advantageous to use three electrodes. There are a variety of montages that can be implemented to produce usable bioelectric signal patterns associated with EEG readings. In general it is desirable to increase the spatial separation between active and reference electrodes since large separations tend to increase signal strength of the EEG. Similar to bioelectric signal patterns associated with EEG readings, a number of considerations determine the optimal electrode placement for bioelectric signal patterns associated with EOG readings using electrode outfitted ear canal inserts. Again, it is desirable to provide a spatial separation of active and reference electrodes such that the electrodes are spaced on opposite sides of the eye, with a spatial separation oriented in the same direction as the expected eye motions (e.g., active and reference electrodes above and below the eye to measure a vertical EOG).

With respect to the third electrode, a ground electrode, there are a number of options for placement. The ground electrode may be positioned within the ear canal, near an outer portion of the ear canal (e.g., between the head and auricle of the ear canal) or on the back of the neck. The potential of both active and reference leads are measured relative to this common ground electrode and only their difference is amplified. Since a subject is capacitively coupled to ground, noise sources common to the active and reference electrodes (e.g., 60 Hz noise) may be significantly reduced. It is therefore desirable to position the ground electrode in a position where it will be able to detect sources of electrical noise common to both the active and reference electrodes, while maintaining a position as far as possible from the active electrode. Thus, the exact placement of the ground electrode is in most cases dependent upon the specific sites chosen for the active and reference electrodes and the electrical interference expected from the surrounding monitoring environment.

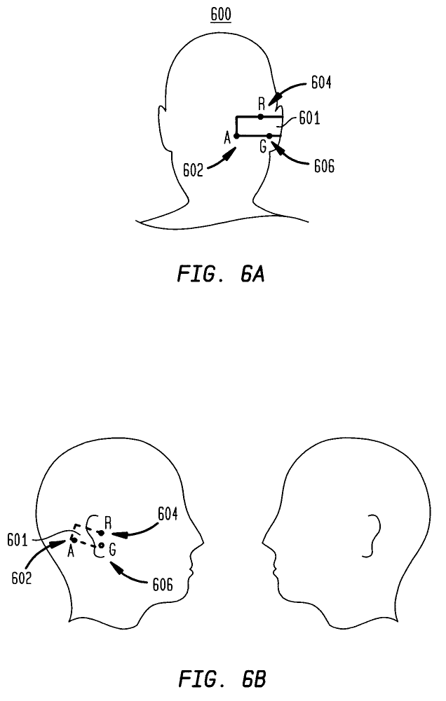

The various electrode placement montages for achieving sufficient spatial separations are illustrated in FIGS. 6-10. In FIG. 6, a back view (FIG. 6A) and side views (FIG. 6B) of an individual equipped with a bioelectrical measurement device is shown. Either measurement device 200 (illustrated in FIGS. 2-4B) or measurement device 500 (FIGS. 5A-5B) may be utilized in the electrode placement montage 600 of FIG. 6. In montage 600, active electrode 602, reference electrode 604 and ground electrode 606 are all provided on a single ear canal insert 601. In this particular arrangement of electrodes, the maximum spatial separation between active electrode 602 and reference electrode 604 is achieved by affixing the electrodes 180 degrees apart along the surface of the ear canal insert 601, as well as spacing electrodes 602 and 604 as far apart laterally along the body of ear canal insert 601. Ground electrode 606 is provided in close proximity to reference electrode 604, but sufficiently distanced from active electrode 602, to detect sources of electrical noise common to both electrodes 602 and 604. Electrode placement montage 600 may be suitable for the measurement of bioelectric signal patterns associated with an EEG reading.

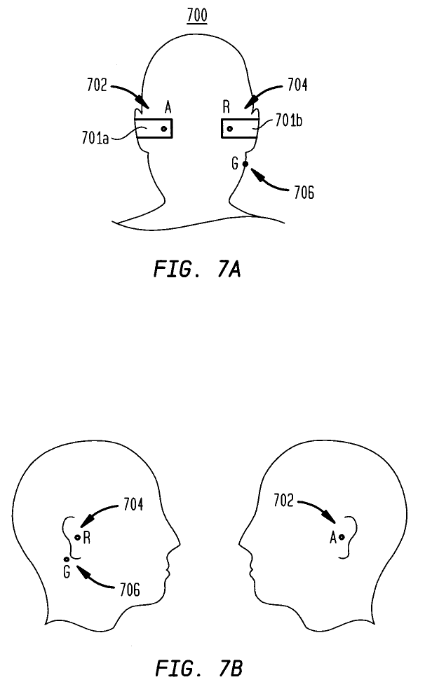

In FIG. 7, a back view (FIG. 7A) and side views (FIG. 7B) of an individual equipped with dual bioelectrical measurement devices (one in each ear) is shown. Either measurement device 200 (illustrated in FIGS. 2-4B), measurement device 500 (FIGS. 5A-5B) or a combination of both (one of each for use in opposing ears) may be utilized in electrode placement montage 700 of FIG. 7. In montage 700, active electrode 702 and reference electrode 704 are provided on separate ear canal inserts 701a and 701b. In this particular arrangement of electrodes, the maximum spatial separation between active electrode 702 and reference electrode 704 is achieved by affixing the electrodes in opposing ear canals. Similar to ground electrode 606 (FIG. 6), ground electrode 706 is provided in close proximity to reference electrode 704, but sufficiently distanced from active electrode 702, to detect sources of electrical noise common to both electrodes 702 and 704. It can be seen from the illustration of this particular arrangement that ground electrode 706 is affixed outside the ear canal against the mastoid portion of the temporal bone behind the auricle of the ear. This may be achieved, for example, by utilizing measurement device 200 having electrode contact 206 provided on the lower inside surface of housing 202 (FIG. 2). Electrode placement montage 700 may be suitable for the measurement of bioelectric signal patterns associated with EEG and horizontal EOG readings.

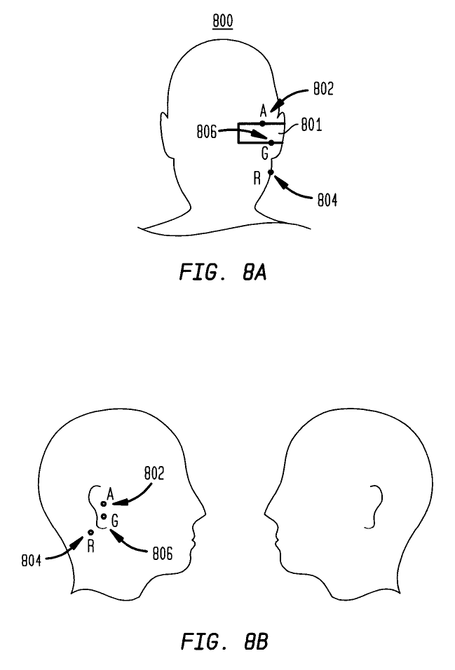

In FIG. 8, a back view (FIG. 8A) and side views (FIG. 8B) of an individual equipped with a bioelectrical measurement device is shown. Here, the use of measurement device 200 is required in the electrode placement montage 800 of FIG. 8. In montage 800, active electrode 802, reference electrode 804 and ground electrode 806 are all provided in close proximity to a single ear canal, wherein active electrode 802 and ground electrode 806 are positioned within the ear canal via ear canal insert 801 and reference electrode 806 is positioned at the mastoid portion of the temporal bone behind the auricle of the ear. Measurement device 200 provides the means for locating electrodes 802, 804 and 806 in this particular manner. Electrode contact 206 may be used as reference electrode 804. Again, in this particular arrangement of electrodes, the maximum spatial separation between active electrode 802 and reference electrode 804 is achieved. Electrode placement montage 800 may be suitable for the measurement of bioelectric signal patterns associated with an EEG reading.

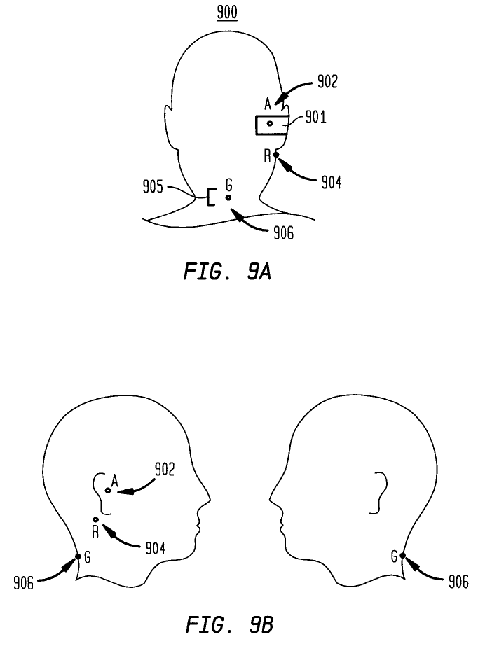

In FIG. 9, a back view (FIG. 9A) and side views (FIG. 9B) of an individual equipped with a bioelectrical measurement device is shown. Similar to montage 800 of FIG. 8, the use of measurement device 200 is preferred in the electrode placement montage 900 of FIG. 9 due to the availability of an electrode on the body of housing 202 for positioning at the mastoid portion of the temporal bone behind the auricle of the ear. In montage 900, active electrode 902, reference electrode 904 and ground electrode 906 are all separated, but kept in close proximity to the ear canal. Active electrode 902 is located in the ear canal via electrode outfitted ear canal insert 901, reference electrode 904 is located at the mastoid portion of the temporal bone behind the auricle of the ear and ground electrode 906 is positioned on the back of the neck. An optional electrode lead (not shown) similar to flexible processing extension 204 may be provided at the base of housing 202 to extend the back of the neck area 905 for connected ground electrode 906. Similar to all previously described montages, the maximum spatial separation between active electrode 902 and reference electrode 904 is achieved. Electrode placement montage 900 may be suitable for the measurement of bioelectric signal patterns associated with an EEG reading.

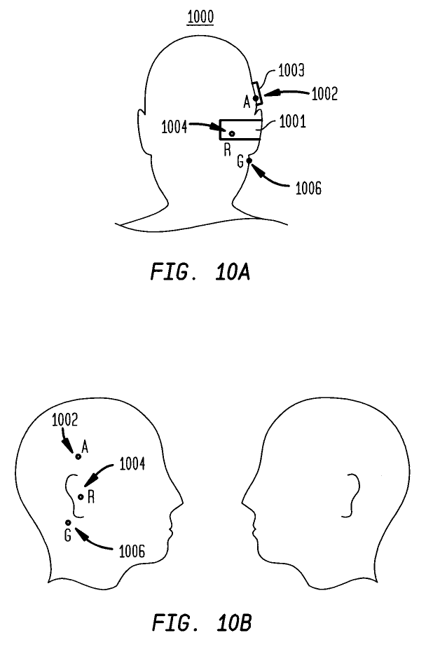

In FIG. 10, a back view (FIG. 10A) and side views (FIG. 10B) of an individual equipped with a bioelectrical measurement device is shown. Similar to montages 800 and 900, the use of measurement device 200 is preferred in electrode placement montage 1000 of FIG. 10 due to the availability of optional electrode contacts 206 and 208 on the body of housing 202 for positioning, respectively, against the mastoid portion of the temporal bone behind the auricle of the ear and a location in proximity to the temple that is above the horizontal plane of the eye. In montage 1000, active electrode 1002, reference electrode 1004 and ground electrode 1006 are all provided in close proximity to the ear canal. Active electrode 1002 is located outside the ear canal proximate to a temple location 1003 crossing above the horizontal plane of the eyes via electrode contact 208. Reference electrode 1004 is positioned within the ear canal via ear canal insert 1001. Ground electrode 1006 is positioned at the mastoid portion of the temporal bone residing behind the auricle of the ear. Measurement device 200 provides the means for locating electrodes 1002, 1004 and 1006 in this particular manner in that electrode contact 208 may be used as active electrode 1002 and electrode contact 206 may be used as ground electrode 1006, thereby providing sites above and below the eye for measuring a vertical EOG reading. Again, in this particular arrangement of electrodes, the maximum spatial separation between active electrode 802 and reference electrode 804 is achieved. Therefore, electrode placement montage 1000 may be suitable for the measurement of bioelectric signal patterns associated with EEG and vertical EOG readings.

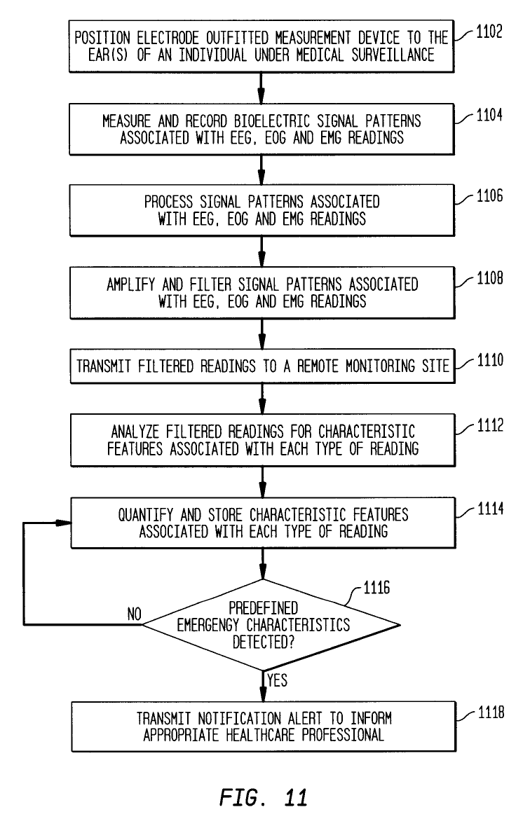

FIG. 11 is an illustrative depiction of the general steps employed by systems 20 and 42 of apparatus 10 for monitoring and analyzing bioelectrical signal patterns of EEG, EOG and EMG readings. In order to initiate the monitoring process of bioelectrical signal patterns at step 1102 the electrodes of measurement device 200, measurement device 500 or alternatively a combination of both must be positioned within and/or in proximity to the ear canal of an individual under medical surveillance. Once properly positioned, electrodes of the corresponding measurement devices may measure detected bioelectrical signal patterns associated with EEG, EOG and EMG readings at step 1104. Detected readings are subsequently processed, at step 1106, by processor 30 provided within electronics unit 24. The processing of detected bioelectrical signal patterns includes converting the detected potentials from analog to digital signals for processing by processor 30. The processing of detected bioelectrical signal patterns additionally includes execution of amplification and appropriate biopotential filtering schemes, at step 1108, in order to distinguish the various bioelectrical readings detected by the electrodes. For example, it is preferable to utilize a biopotential filtering scheme for separating and EMG, EOG and EMG readings detected by the electrodes.

Processed bioelectrical signal patterns may then be temporarily stored in memory component 40 (FIG. 1) to be transmitted, at step 1110, to a remote monitoring station 42. Additional processing may be performed by processor 50 of user computer 48 at remote monitoring system 42. Alternatively, steps 1106 and 1108 may be performed after transmission step 1110 by incorporating similar processing components provided in electronics unit 24 with processor 50 of user computer 48, thereby transmitting raw bioelectrical signal pattern data from system 20 to system 42 to be similarly processed.

Filtered EEG, EOG and EMG readings are analyzed at step 1112. The filtered readings may be displayed via display interface 52 of user computer 48 (FIG. 1). These filtered readings associated with the transmitting individual are quantified and stored at step 1114, for example, in memory component 55. Alternatively, bioelectric related data for a patient under constant medical surveillance may be stored at a remote location. If a predefined emergency characteristic is detected in any of the analyzed and quantified readings at step 1116, system 42 may be directed to activate notification interface 54, thereby transmitting, at step 1118, the appropriate data and/or predefined notification alarms and messages via transceiver 44 to a medically enabled mobile device 56. A healthcare professional (e.g., an individual's personal physician) may then effectively analyze and prescribe the appropriate action to be taken. For example, an individual's primary physician may receive an alert and corresponding images of an abnormal bioelectrical signal pattern via a medically enabled PDA device 56c. It should be understood that the notification procedures described above are provided merely as examples and that various notification procedures may be implemented in accordance with the principles of the invention.

It can be seen from the aforementioned description that brain activity can be monitored without need for invasive sensors. However, the bioelectric measurement device described in the present invention is not only limited to providing a wirelessly enabled monitoring means of ambulatory individuals, but is also configured to evoke brain activity in response to predetermined stimuli for purposes of monitoring brain function and associated physiological states or diseases for particular individuals that would most benefit from such surveillance.

It is well understood that specific sensory stimulation may be presented to an individual in order to evoke brain activity. The brain activity that is generated in response to a known stimulus is called an evoked potential, particularly auditory evoked potentials (AEP), which is a well known means for evoking potential to monitor states of consciousness. The AEP of an EEG reading, for example, may be measured by presenting acoustic stimulation to an individual under medical surveillance and recording the corresponding EEG reading. The EEG that is elicited in response to the acoustic stimulus is then analyzed for characteristic features that are linked to the state of consciousness. These features are ultimately quantified to provide clinically useful bioelectric signal pattern measurements of an EEG reading.

In the present invention, bioelectric measurement devices are equipped to induce an AEP, as well as measure the corresponding bioelectric signal patterns (as previously described). For example, electronics unit 24 of FIG. 1 is equipped with auditory component 38 and speaker 26 and may be integrated within exterior housing 202 of measurement device 200 or housing 501 of measurement device 500. These electronic components in combination provide a means for producing an acoustic stimulus within the ear canal in order to induce an AEP and provide a bioelectrical signal pattern for measurement by electrodes strategically placed within and in close proximity to the ear canal.

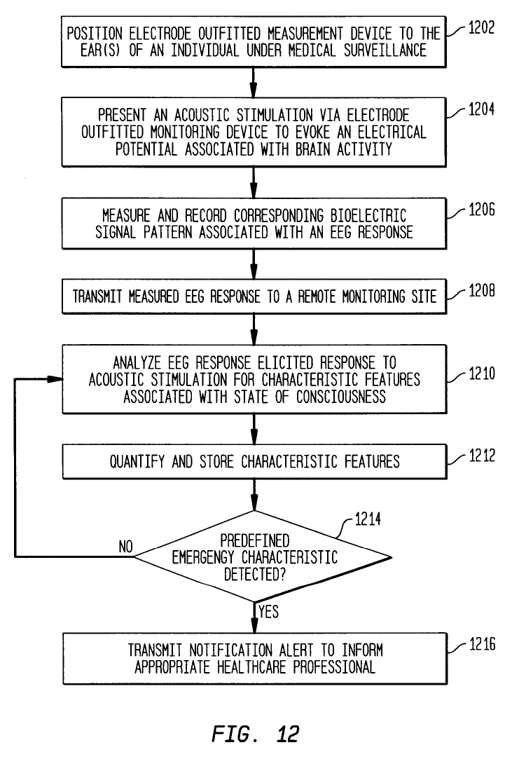

An illustrative depiction of the general steps employed by systems 20 and 42 of apparatus 10 for inducing an AEP and analyzing the corresponding bioelectrical signal patterns of an EEG reading is described with reference to the flowchart of FIG. 12. The monitoring of bioelectrical signal patterns associated with an EEG reading generated in response to an AEP is initiated by first positioning, at step 1202, the electrodes of measurement device 200, measurement device 500 or alternatively a combination of both within and/or in proximity to the ear canal of an individual under medical surveillance. When the select measurement device and its electrodes are properly positioned, an acoustic stimulation is presented, at step 1204, into the ear canal of the individual under surveillance via speaker 26. The acoustic stimulation may be preprogrammed for specific time presentations or initiated on demand from a remote site via a wireless transmission of commands to the select measurement device. Presentation of acoustic stimulation may be regulated by auditory component 38 of electronics unit 24 provided within the select measurement device attached to the individual under medical surveillance. The acoustic stimulation may be a vocal dictation, tone or any other applicable sound for stimulating brain activity.

At step 1206, bioelectric signal patterns associated with an EEG reading produced in response to the acoustic stimulation provided at step 1204 are appropriately measured and recorded. Measured signal patterns may be associated and recorded in synchrony with the acoustic stimulation and transmitted, at step 1208, to a remote monitoring site for additional processing and analyzing at step 1210. It is often important to measure latency time periods between acoustic stimulation and events evoked in an EEG reading. It is also important to perform mathematical averaging of a series of EEG responses to repeated acoustic stimulation to improve signal-to-noise characteristics of the bioelectric signal pattern. Therefore, the processing and analyzing that occurs at step 1210 may utilize processor 50 of remote monitoring system 42 to implement electronic processing means for synchronizing the collection of bioelectrical data received at the electrodes of the measurement device with the time of acoustic stimulation. Thereafter, at step 1212, further analysis means are provided to quantify features of the evoked bioelectrical signal patterns associated with an EEG reading in order to provide a measure of clinically relevant quantity representing level of consciousness.

Abnormal EEG responses to acoustic stimulation may be detected at step 1214. Known features indicative of abnormality and or emergency situations may be predefined and associated with a set of medically responsive procedures to be executed upon detection. One such response may be triggering of an alarm and transmission of a notification alert to the appropriate healthcare professional, as provided at step 1216. Healthcare professionals may receive such notifications on medically enabled mobile devices 56 via a wireless transmission 60 received at antenna 58. However, the aforementioned response procedure is provided merely as an example. Alternative notification procedures may be implemented and is well within the scope of the present invention.

One skilled in the art will appreciate that the present invention can be practiced by other than the described embodiments, which are presented for purposes of illustration and not by way of limitation, and the present invention is limited only by the claims that follow.

What is claimed is:

1. A bioelectric measurement and monitoring apparatus, comprising:
an active electrode, a reference electrode and a ground electrode;
at least one measurement device having at least one of said electrodes configured for placement within an ear canal;
at least one remote device adapted to process and monitor bioelectric related data; and
a wireless transmission means for communicating said bioelectric related data between said at least one measurement device and said at least one remote device.

2. The bioelectric measurement and monitoring apparatus of claim 1, further comprising at least one medically enabled mobile device adapted to receive said bioelectric related data via said wireless transmission means.

3. The bioelectric measurement and monitoring apparatus of claim 1, wherein said measurement device is constructed for placement entirely within said ear canal.

4. The bioelectric measurement and monitoring apparatus of claim 3, wherein said measurement device constructed for placement entirely within said ear canal houses electronic components for processing signals measures via said electrodes.

5. The bioelectric measurement and monitoring apparatus of claim 1, wherein a first portion of said measurement device is constructed for placement within said ear canal, and wherein a second portion of said measurement device is constructed for placement outside of said ear canal, said second portion being positioned between an auricle of an ear and corresponding side of a head of an individual.

6. The bioelectric measurement and monitoring apparatus of claim 5, wherein said second portion of said measurement device houses electronic components for processing bioelectric related data and is coupled to said first portion via a flexible processing extension.

7. The bioelectric measurement and monitoring apparatus of claim 5, wherein said second portion of said measurement device further comprises at least one of said electrodes affixed to an outside surface of its housing.

8. The bioelectric measurement and monitoring apparatus of claim 7, wherein said at least one electrode affixed to said outside surface of said housing of said second portion of said measurement device is configured to make contact in close proximity to a mastoid portion of a temporal bone residing behind said auricle of said ear.

9. The bioelectric measurement and monitoring apparatus of claim 7, wherein said at least one electrode affixed to said outside surface of said housing of said second portion of said measurement device is configured to make contact in close proximity to a temple position crossing above a horizontal plane of the eyes.

10. The bioelectric measurement and monitoring apparatus of claim 1, wherein said active electrode is positioned within said ear canal, in close proximity to a mastoid portion of a temporal bone residing behind said auricle of said ear or in close proximity to a temple position crossing above a horizontal plane of the eyes.

11. The bioelectric measurement and monitoring apparatus of claim 1, wherein said reference electrode is positioned within said ear canal, in close proximity to a mastoid portion of a temporal bone residing behind said auricle of said ear or in close proximity to a temple position crossing above a horizontal plane of the eyes.

12. The bioelectric measurement and monitoring apparatus of claim 1, wherein said ground electrode is positioned within said ear canal, in close proximity to a mastoid portion of a temporal bone residing behind said auricle of said ear, in close proximity to a temple position crossing above a horizontal plane of the eyes or on the back of a neck.

13. The bioelectric measurement and monitoring apparatus of claim 1, wherein said remote device is a user computer having a receiving and transmitting means, a processing means, a display interface, a notification interface and a memory component.

14. The bioelectric measurement and monitoring apparatus of claim 13, wherein said notification interface is configured to transmit via said transmitting means an alert notification and corresponding bioelectric related data triggering said transmission of said alert notification to a medically enabled mobile device.

15. A method for measuring and monitoring bioclectric signal patterns, comprising the steps of:
affixing an active electrode, a reference electrode and a ground electrode in close proximity to an ear canal, wherein at least one of said electrodes is positioned within said ear canal;
measuring said bioelectric signal patterns using said electrodes; and
processing said bioelectric signal patterns.

16. The method of claim 15, further comprising wirelessly transmitting bioelectric related data associated with said measured bioelectric signal patterns to a remote monitoring device.

17. The method of claim 15, further comprising identifying characteristic features in bioelectric related data associated with said measured bioelectric signal patterns.

18. The method of claim 17, wherein said identified characteristic features in said bioelectric related data are used to determine the presence of anomalies or abnormalities in said measured bioelectric signal patterns.

19. The method of claim 17, wherein said identified characteristic features in said bioelectric related data is quantified and stored to provide a clinically useful measurement of said bioelectric signal patterns.

20. The method of claim 15, further comprising presenting an auditory stimulus into said ear canal to evoke said bioelectrical signal patterns associated with brain activity.

21. The method of claim 15, further comprising providing notifications related to said measured bioelectric signal patterns.

22. The method of claim 21, wherein said notifications are wirelessly transmitted to a mobile device.

23. The method of claim 22, wherein said wirelessly transmitted notifications are accompanied by said bioelectric related data for display on a medically enabled mobile device.
